(19) United States
(12) Patent Application Publication
(10) Pub. No.: US 2026/0250356 A1
(43) Pub. Date: Aug. 27, 2026

(54) COST-EFFECTIVE METHOD FOR EXTRACTING FISH SCALE COLLAGEN HAVING LARGE MOLECULAR WEIGHT

(71) Applicant: Hangzhou Singclean Medical Products Co., Ltd, Hangzhou (CN)

(72) Inventors: Jiaqi Hu, Hangzhou (CN); Peilin Wei, Hangzhou (CN); Minxin Yang, Hangzhou (CN); Qiulin He, Hangzhou (CN); Wei Huang, Hangzhou (CN); Junjie Fan, Hangzhou (CN)

(21) Appl. No.: 19/221,469

(22) Filed: May 28, 2025

(30) Foreign Application Priority Data
Feb. 24, 2025 (CN) 202510205518.7

(51) Int. Cl. C07K 14/78; C07K 1/14; C07K 1/34; C07K 1/36

(52) U.S. Cl. CPC C07K 14/78; C07K 1/145; C07K 1/34; C07K 1/36

(57) ABSTRACT

The present disclosure provides a cost-effective method for extracting fish scale collagen having a large molecular weight, pertaining to the technical field of fish scale collagen. The present disclosure includes: 1) performing pretreatment, including: repeatedly subjecting fish scales to frozen storage and thawing; 2) performing impurity protein removal, including: removing fish silver from the pretreated fish scales; and soaking the fish scales in sodium chloride solution, followed by cleaning and drying; 3) performing fish scale collagen extraction, including: mixing the dried fish scales with purified water according to a ratio of 1:5 to 1:20, subjecting the mixture to hydrothermal extraction at 40° C. to 100° C. for 1 to 4 hours, performing solid-liquid separation, and collecting the solution as a fish scale collagen extract; and 4) lyophilizing the fish scale collagen extract to obtain a solid fish scale collagen product.

### CROSS-REFERENCE TO RELATED APPLICATION

The present application claims priority from Chinese Patent Application No. 202510205518.7 filed on Feb. 24, 2025, the contents of which are incorporated herein by reference in their entirety.

### TECHNICAL FIELD

The present disclosure relates to the technical field of animal collagen, and specifically to a cost-effective method for extracting fish scale collagen having a large molecular weight.

### BACKGROUND

As the main component of extracellular matrix, collagen is a very important protein in human body. It can supplement the nutrition needed by all layers of skin, and has the effects of moisturizing skin, delaying aging, and enhancing beauty. Traditionally, collagen is mainly extracted from the skins and bones of terrestrial animals such as cattle and pigs. However, due to the outbreak of mad cow disease, foot-and-mouth disease, and other diseases, people become concerned about the safety of traditional collagen products. With the increasing demand for collagen products in the fields such as medicine and cosmetics, there is an urgent need for safe and reliable collagen raw materials.

Studies have shown that fish scales are rich in protein and fat, among which the collagen content is up to 45%, so fish scale collagen has become one of the popular candidates for mammalian collagen substitutes. In recent years, an increasing number of studies have been reported on the extraction of collagen from fish scales. However, in most of the studies, fish scales are pretreated with strong acid and alkali before extraction. The excessive use of strong acid and alkali not only causes environmental pollution, but also damages the complete structure of collagen, which is not conducive to collagen extraction. In addition, although various methods for extracting collagen from fish scales have been proposed, such methods have their respective advantages and disadvantages. For example, an acid-enzyme method can retain the molecular weight and complete structure of collagen, but requires a complicated procedure in large-scale production, resulting in high costs. A conventional hydrothermal method is easy to operate, but has low low-temperature extraction efficiency. In order to improve the yield, high-temperature extraction at, for example, 100° C., is often used. Although high-temperature extraction can improve the yield, the high temperature will damage the triple helix structure of collagen since collagen is thermal-sensitive. Studies have shown that only collagen that retains the complete protein structure and has an appropriate molecular weight can achieve a desirable effect. In order to achieve a balance between extraction efficiency, molecular weight, and integrity of protein structure, a gradient hydrothermal method for extracting fish scale collagen is devised.

The prior art has the following technical problems.
1) The methods for extracting fish scale collagen in the prior art involve a complicated procedure and high costs. Although fish scale collagen with high purity and good water solubility can be obtained by using different enzymes and permeable membranes, the high costs of enzyme preparations and the complicated enzyme procedure lead to high costs in large-scale production, limiting the range of applications of fish scale collagen.
2) The pre-treatment procedure in the prior art is complicated, which is not conducive to the extraction of fish scale collagen. Although the soaking of fish scales with an acid or alkali solution can remove impurities and weaken the dense fibrous structure to facilitate the extraction of fish scale collagen, the waste liquid generated will cause environmental pollution, and the solution residue will cause the breakdown of fish scale collagen, which is not conducive to large-scale production.
3) The fish scale collagen extracted by the conventional hydrothermal method has a serious degree of denaturation. Although a high yield of fish scale collagen can be obtained by increasing the temperature and prolonging the treatment time, the high temperature will cause the triple helix structure of fish scale collagen to dissociate into free polypeptide chains since fish scale collagen is thermal-sensitive, resulting in a dispersed molecular weight distribution.
4) Fish scale collagen extracted in different batches using the conventional hydrothermal method have different molecular weights and an incomplete protein structure.

### SUMMARY

To solve the problems in the prior art, the present disclosure provides a cost-effective method for extracting fish scale collagen having a large molecular weight.

To solve the above problems, the present disclosure is mainly accomplished through the following technical solutions.

The present disclosure discloses a cost-effective method for extracting fish scale collagen having a large molecular weight, including:
1) performing pretreatment, comprising: collecting and packaging fish scales, followed by repeated frozen storage and thawing to obtain pretreated fish scales;
2) performing impurity protein removal, comprising: removing fish silver from the pretreated fish scales obtained in the step 1) and cleaning with pure water; and soaking the cleaned fish scales in a salt solution to remove impurity proteins, rinsing the fish scales with purified water until clear, followed by drying;
3) performing fish scale collagen extraction, comprising: mixing the dried fish scales obtained in the step 2) with purified water according to a ratio of 1:5 to 1:20, subjecting the mixture to hydrothermal extraction at 40° C. to 100° C. for 1 to 4 hours, performing solid-liquid separation, and collecting the solution as a fish scale collagen extract; and
4) pre-cooling and lyophilizing the fish scale collagen extract collected in the step 3) to obtain a solid fish scale collagen product.

As a further improvement, in the step 1) of the present disclosure, the frozen storage temperature of the packaged fish scales is −80° C. to −20° C.

As a further improvement, in the step 1) of the present disclosure, the temperature of thawing is 25° C. to 45° C., the time of thawing is 0.5 hours to 3 hours, and the number of times that the frozen storage and thawing operations are repeated is 1 to 3. In the present disclosure, the temperature difference between frozen storage and thawing is large, the temperature fluctuation generated in the implementation process causes ice crystals inside the fish scales to be more easily recrystallized, and the accumulation and expansion of ice crystals can more easily destroy the internal tissue structure, facilitating the subsequent extraction of collagen. In the present disclosure, the frozen storage and thawing operations are repeated multiple times, to completely destroy the interaction force inside the fish scales, thereby reducing the energy consumption of subsequent fish scale collagen extraction.

As a further improvement, in the step 2) of the present disclosure, the salt solution is any one of sodium chloride solution and sodium carbonate solution, the mass fraction of the salt solution is 5% to 20%, and the soaking time is 0.5 hours to 2 hours. In the present disclosure, the fish scales are soaked in the salt solution and thus treated under mild conditions, so impurity proteins can be removed while preventing the structural integrity of collagen from being destroyed. The sodium chloride solution may be prepared on site, which is simple to operate, has low energy consumption, and is convenient to use.

As a further improvement, in the step 2) of the present disclosure, the fish scales are dried at 20° C. to 65° C. for 3 hours to 24 hours. Because the fish scale collagen is sensitive to ambient temperature and the fish scale collagen will be inactivated when the temperature is too high, using such a drying method in the present disclosure can ensure that the fish scales are completely dehydrated without destroying the properties of the fish scale collagen.

As a further improvement, in the step 3) of the present disclosure, the hydrothermal extraction of fish scale collagen is a gradient hydrothermal extraction process, with gradient temperature intervals of 40° C. to 70° C., 70° C. to 90° C., and 90° C. to 100° C., and with the gradient temperature change being a gradient temperature increase or a gradient temperature decrease.

As a further improvement, in the step 3) of the present disclosure, the ratio of the fish scales to water is 1:5 to 1:10, the extraction time at 40° C. to 70° C. is 0.5 hours to 2 hours, the extraction time at ° C. 70 to 90° C. is 0.5 hours to 1 hour, and the extraction time at 90° C. to 100° C. is 0.5 hours to 2 hours.

As a further improvement, in the step 3) of the present disclosure, the solid-liquid separation is performed by filtration or centrifugation, and the fish scale collagen extract is collected by stages or cumulatively. In the present disclosure, collection by stages is to respectively collect the fish scale collagen extracts obtained in the corresponding gradient temperature ranges according to the ratio of the fish scales to purified water. Cumulative collection is to collect the fish scale collagen extract obtained by gradient hydrothermal extraction at a time.

As a further improvement, in the step 4) of the present disclosure, the fish scale collagen extract is dried by any one of vacuum freeze-drying, spray freeze-drying, gel drying, and vacuum drying. The lyophilized collagen product prepared by the present disclosure has higher stability than a collagen solution.

As a further improvement, the solid fish scale collagen product prepared by the method is in the form of powder, granules, or sponge. Solid fish scale collagen products having a large molecular weight in various forms such as powder and sponge can be obtained from fish scales by using the preparation process of the present disclosure, and such products can be widely applied to the fields such as medicine and cosmetics.

In the above technical solutions, the cost-effective method for extracting fish scale collagen having a large molecular weight according to the present disclosure has the following beneficial effects.
1. The preparation process provided by the present disclosure avoids the use of an enzyme and a permeable membrane to screen out collagen having a specific molecular weight, and therefore, is convenient to operate and reduces the costs of collagen extraction.
2. The present disclosure uses the salt solution and the process in which the temperature ranges are 40° C. to 70° C., 70° C. to 90° C., and 90° C. to 100° C. and the temperature change is a gradient temperature increase or a gradient temperature decrease, thereby avoiding the damage of the complete structure of collagen by strong acid or alkali solution and long-time high-temperature cooking.
3. By the process where the temperature ranges are 40° C. to 70° C., 70° C. to 90° C., and 90° C. to 100° C., the extraction time at 40° C. to 70° C. is 0.5 hours to 2 hours, the extraction time at 70° C. to 90° C. is 0.5 hours to 1 hour, the extraction time at 90° C. to 100° C. is 0.5 hours to 2 hours, and the temperature change is a gradient temperature increase or a gradient temperature decrease, the molecular weight distribution is more concentrated and the structure of collagen is more complete while retaining high extraction efficiency. A conventional hydrothermal method is to directly immerse the raw materials having been subjected to certain pretreatment in aqueous solution at a high temperature to obtain collagen. Although the solubility of collagen increases with the increase of temperature, the long-time high-temperature cooking process leads to a more thorough dissociation of the triple helix structure into free polypeptide chains and therefore a more dispersed molecular weight distribution. The gradient hydrothermal method provides a low-temperature buffer zone to slow down the dissociation of the triple helix while ensuring a high extraction rate, and collagen refolds at low temperature, thus retaining a more complete collagen structure and achieving a concentrated molecular weight distribution.
4. The gradient temperature control process is used to reduce the differences between batches, thus obtaining homogeneous and complete collagen.

### BRIEF DESCRIPTION OF THE DRAWINGS

FIG. 1 is a diagram showing the Fourier Transform Infrared (FTIR) spectroscopy results of fish scale collagen in comparative examples;

FIG. 2 is a diagram showing the FTIR spectroscopy results of fish scale collagen in examples; and

FIG. 3 is a graph showing the SDS-PAGE gel electrophoresis results of fish scale collagen.

### DETAILED DESCRIPTION

To make the objectives, technical solutions, and advantages of the present disclosure more comprehensible, a detailed description will be given below with reference to specific examples of the present disclosure. The examples described are merely some examples, rather than all of the examples of the present disclosure. All other examples obtained by those of ordinary skill in the art without creative efforts based on the examples of the present disclosure shall fall within the protection scope of the present disclosure.

### Example 1

1) Pretreatment: Fish scales were collected and packaged, followed by frozen storage at −20° C. and thawing at 25° C. for 1 hour. The frozen storage and thawing operations were repeated three times to obtain pretreated fish scales.
2) Impurity protein removal: Fish silver was removed from the pretreated fish scales, and the resulting fish scales were rinsed with purified water until clear. The fish scales were soaked in 10% sodium chloride solution and allowed to stand for 1 hour. Finally, the fish scales were taken out, washed, and dried at 25° C. for later use.
3) Fish scale collagen extraction: First, the dried fish scales were mixed with purified water according to a ratio of 1:7.5, and the mixture was subjected to hydrothermal extraction at 55° C. for 1.5 hours. Then, purified water was added according to a ratio of the fish scales to purified water being 1:5, and the mixture was subjected to hydrothermal extraction at 75° C. for 1 hour. Finally, purified water was added according to a ratio of the fish scales to purified water being 1:7.5, and the mixture was subjected to hydrothermal extraction at 95° C. for 1.5 hours. The extraction was performed for 4 hours in total. After solid-liquid separation, a fish scale collagen extract was collected cumulatively.
4) The fish scale collagen extract was pre-cooled at −20° C. and lyophilized in a lyophilizer to obtain a solid fish scale collagen product.

### Example 2

1) Pretreatment: Fish scales were collected and packaged, followed by frozen storage at −20° C. and thawing at 25° C. for 1 hour. The frozen storage and thawing operations were repeated three times to obtain pretreated fish scales.
2) Impurity protein removal: Fish silver was removed from the pretreated fish scales, and the resulting fish scales were rinsed with purified water until clear. The fish scales were soaked in 10% sodium chloride solution and allowed to stand for 1 hour. Finally, the fish scales were taken out, washed, and dried at 25° C. for later use.
3) Fish scale collagen extraction: First, the dried fish scales were mixed with purified water according to a ratio of 1:7.5, and the mixture was subjected to hydrothermal extraction at 95° C. for 1.5 hours. Then, purified water was added according to a ratio of the fish scales to purified water being 1:5, and the mixture was subjected to hydrothermal extraction at 75° C. for 1 hour. Finally, purified water was added according to a ratio of the fish scales to purified water being 1:7.5, and the mixture was subjected to hydrothermal extraction at 55° C. for 1.5 hours. The extraction was performed for 4 hours in total. After solid-liquid separation, a fish scale collagen extract was collected cumulatively.
4) The fish scale collagen extract was pre-cooled at −20° C. and lyophilized in a lyophilizer to obtain a solid fish scale collagen product.

### Example 3

1) Pretreatment: Fish scales were collected and packaged, followed by frozen storage at −20° C. and thawing at 25° C. for 1 hour. The frozen storage and thawing operations were repeated three times to obtain pretreated fish scales.
2) Impurity protein removal: Fish silver was removed from the pretreated fish scales, and the resulting fish scales were rinsed with purified water until clear. The fish scales were soaked in 10% sodium chloride solution and allowed to stand for 1 hour. Finally, the fish scales were taken out, washed, and dried at 25° C. for later use.
3) Fish scale collagen extraction: First, the dried fish scales were mixed with purified water according to a ratio of 1:7.5, and the mixture was subjected to hydrothermal extraction at 55° C. for 1.5 hours. Solid-liquid separation was performed, and a first fish scale collagen extract was collected. Then, the solid was mixed with purified water according to a ratio of 1:5, and the mixture was subjected to hydrothermal extraction at 75° C. for 1 hour. Solid-liquid separation was performed, and a second fish scale collagen extract was collected. Finally, the solid was mixed with purified water according to a ratio of 1:7.5, and the mixture was subjected to hydrothermal extraction at 95° C. for 1.5 hours. Solid-liquid separation was performed, and a third fish scale collagen extract was collected. The extraction was performed for 4 hours in total. The three fish scale collagen extracts collected were combined.
4) The combined fish scale collagen extract was pre-cooled at −20° C. and lyophilized in a lyophilizer to obtain a solid fish scale collagen product.

### Example 4

1) Pretreatment: Fish scales were collected and packaged, followed by frozen storage at −20° C. and thawing at 25° C. for 1 hour. The frozen storage and thawing operations were repeated three times to obtain pretreated fish scales.
2) Impurity protein removal: Fish silver was removed from the pretreated fish scales, and the resulting fish scales were rinsed with purified water until clear. The fish scales were soaked in 10% sodium chloride solution and allowed to stand for 1 hour. Finally, the fish scales were taken out, washed, and dried at 25° C. for later use.
3) Fish scale collagen extraction: First, the dried fish scales were mixed with purified water according to a ratio of 1:7.5, and the mixture was subjected to hydrothermal extraction at 95° C. for 1.5 hours. Solid-liquid separation was performed, and a first fish scale collagen extract was collected. Then, the solid was mixed with purified water according to a ratio of 1:5, and the mixture was subjected to hydrothermal extraction at 75° C. for 1 hour. Solid-liquid separation was performed, and a second fish scale collagen extract was collected. Finally, the solid was mixed with purified water according to a ratio of 1:7.5, and the mixture was subjected to hydrothermal extraction at 55° C. for 1.5 hours. Solid-liquid separation was performed, and a third fish scale collagen extract was collected. The extraction was performed for 4 hours in total. The three fish scale collagen extracts collected were combined.
4) The combined fish scale collagen extract was pre-cooled at −20° C. and lyophilized in a lyophilizer to obtain a solid fish scale collagen product.

### Example 5

1) Pretreatment: Fish scales were collected and packaged, followed by frozen storage at −20° C. and thawing at 25° C. for 1 hour. The frozen storage and thawing operations were repeated three times to obtain pretreated fish scales.
2) Impurity protein removal: Fish silver was removed from the pretreated fish scales, and the resulting fish scales were rinsed with purified water until clear. The fish scales were soaked in 10% sodium chloride solution and allowed to stand for 1 hour. Finally, the fish scales were taken out, washed, and dried at 25° C. for later use.
3) Fish scale collagen extraction: First, the dried fish scales were mixed with purified water according to a ratio of 1:7.5, and the mixture was subjected to hydrothermal extraction at 60° C. for 0.5 hours. Then, purified water was added according to a ratio of the fish scales to purified water being 1:5, and the mixture was subjected to hydrothermal extraction at 80° C. for 1 hour. Finally, purified water was added according to a ratio of the fish scales to purified water being 1:7.5, and the mixture was subjected to hydrothermal extraction at 100° C. for 2.5 hours. The extraction was performed for 4 hours in total. After solid-liquid separation, a fish scale collagen extract was collected cumulatively.
4) The fish scale collagen extract was pre-cooled at −20° C. and lyophilized in a lyophilizer to obtain a solid fish scale collagen product.

### Example 6

1) Pretreatment: Fish scales were collected and packaged, followed by frozen storage at −20° C. and thawing at 25° C. for 1 hour. The frozen storage and thawing operations were repeated three times to obtain pretreated fish scales.
2) Impurity protein removal: Fish silver was removed from the pretreated fish scales, and the resulting fish scales were rinsed with purified water until clear. The fish scales were soaked in 10% sodium chloride solution and allowed to stand for 1 hour. Finally, the fish scales were taken out, washed, and dried at 25° C. for later use.
3) Fish scale collagen extraction: First, the dried fish scales were mixed with purified water according to a ratio of 1:7.5, and the mixture was subjected to hydrothermal extraction at 60° C. for 1 hour. Then, purified water was added according to a ratio of the fish scales to purified water being 1:5, and the mixture was subjected to hydrothermal extraction at 80° C. for 1 hour. Finally, purified water was added according to a ratio of the fish scales to purified water being 1:7.5, and the mixture was subjected to hydrothermal extraction at 100° C. for 2 hours. The extraction was performed for 4 hours in total. After solid-liquid separation, a fish scale collagen extract was collected cumulatively.
4) The fish scale collagen extract was pre-cooled at −20° C. and lyophilized in a lyophilizer to obtain a solid fish scale collagen product.

### Example 7

1) Pretreatment: Fish scales were collected and packaged, followed by frozen storage at −20° C. and thawing at 25° C. for 1 hour. The frozen storage and thawing operations were repeated three times to obtain pretreated fish scales.
2) Impurity protein removal: Fish silver was removed from the pretreated fish scales, and the resulting fish scales were rinsed with purified water until clear. The fish scales were soaked in 10% sodium chloride solution and allowed to stand for 1 hour. Finally, the fish scales were taken out, washed, and dried at 25° C. for later use.
3) Fish scale collagen extraction: First, the dried fish scales were mixed with purified water according to a ratio of 1:7.5, and the mixture was subjected to hydrothermal extraction at 60° C. for 2 hours. Then, purified water was added according to a ratio of the fish scales to purified water being 1:5, and the mixture was subjected to hydrothermal extraction at 80° C. for 1 hour. Finally, purified water was added according to a ratio of the fish scales to purified water being 1:7.5, and the mixture was subjected to hydrothermal extraction at 100° C. for 1 hour. The extraction was performed for 4 hours in total. After solid-liquid separation, a fish scale collagen extract was collected cumulatively.
4) The fish scale collagen extract was pre-cooled at −20° C. and lyophilized in a lyophilizer to obtain a solid fish scale collagen product.

### Example 8

1) Pretreatment: Fish scales were collected and packaged, followed by frozen storage at −20° C. and thawing at 25° C. for 1 hour. The frozen storage and thawing operations were repeated three times to obtain pretreated fish scales.
2) Impurity protein removal: Fish silver was removed from the pretreated fish scales, and the resulting fish scales were rinsed with purified water until clear. The fish scales were soaked in 10% sodium chloride solution and allowed to stand for 1 hour. Finally, the fish scales were taken out, washed, and dried at 25° C. for later use.
3) Fish scale collagen extraction: First, the dried fish scales were mixed with purified water according to a ratio of 1:7.5, and the mixture was subjected to hydrothermal extraction at 60° C. for 2.5 hours. Then, purified water was added according to a ratio of the fish scales to purified water being 1:5, and the mixture was subjected to hydrothermal extraction at 80° C. for 1 hour. Finally, purified water was added according to a ratio of the fish scales to purified water being 1:7.5, and the mixture was subjected to hydrothermal extraction at 100° C. for 0.5 hours. The extraction was performed for 4 hours in total. After solid-liquid separation, a fish scale collagen extract was collected cumulatively.
4) The fish scale collagen extract was pre-cooled at −20° C. and lyophilized in a lyophilizer to obtain a solid fish scale collagen product.

### Comparative Example 1

Step 3) in Example 1 was replaced with: mixing the dried fish scales with purified water according to a ratio of 1:20, subjecting the mixture to hydrothermal extraction at 55° C. for 4 hours, performing solid-liquid separation, and collecting a fish scale collagen extract. The other steps were the same as those in Example 1.

### Comparative Example 2

Step 3) in Example 1 was replaced with: mixing the dried fish scales with purified water according to a ratio of 1:20, subjecting the mixture to hydrothermal extraction at 75° C. for 4 hours, performing solid-liquid separation, and collecting a fish scale collagen extract. The other steps were the same as those in Example 1.

### Comparative Example 3

Step 3) in Example 1 was replaced with: mixing the dried fish scales with purified water according to a ratio of 1:20, subjecting the mixture to hydrothermal extraction at 95° C. for 4 hours, performing solid-liquid separation, and collecting a fish scale collagen extract. The other steps were the same as those in Example 1.

In the present disclosure, a biuret method was used to determine the protein content in the solid product and measure the extraction rate of the preparation process.

Test method: 1) A bovine serum albumin reference solution was prepared according to 10 mg/mL. 0 mL, 0.05 mL, 0.1 mL, 0.2 mL, 0.4 mL, 0.6 mL, 0.8 mL, and 1.0 mL of the reference solution were accurately measured and respectively placed in test tubes, followed by addition of water to make the volume to 1.0 mL. 4.0 mL of biuret test solution was added to each of the bovine serum albumin reference solutions having different concentrations prepared using the above method, mixed until uniform at room temperature, allowed to stand for 30 minutes, and measured for the absorbance at a wavelength of 540 nm. A standard curve was plotted. 2) An appropriate amount of the sample was added to water to prepare a sample solution. 4.0 mL of biuret test solution was added to 1 mL of the sample solution, mixed until uniform at room temperature, allowed to stand for 30 minutes, and measured for the absorbance at a wavelength of 540 nm. The protein content in the sample was calculated according to the standard curve, and finally converted to the extraction rate.

The solid products prepared in the examples and comparative examples of the present disclosure were tested for collagen content. The results are shown in Table 1.

TABLE 1
Results of extraction rate of fish scale collagen

| Case | Temperature (° C.) | Time (h) | Collection mode | Extraction rate (%) |
|---|---|---|---|---|
| Example 1 | 55 − 75 − 95 | 1.5-1-1.5 | Cumulative collection | 32.76 |
| Example 2 | 95 − 75 − 55 | 1.5-1-1.5 | Cumulative collection | 33.14 |
| Example 3 | 55 + 75 + 95 | 1.5-1-1.5 | Collection by stages | 25.86 |
| Example 4 | 95 + 75 + 55 | 1.5-1-1.5 | Collection by stages | 28.83 |
| Example 5 | 60 − 80 − 100 | 0.5-1-2.5 | Cumulative collection | 19.59 |
| Example 6 | 60 − 80 − 100 | 1-1-2 | Cumulative collection | 20.56 |
| Example 7 | 60 − 80 − 100 | 2-1-1 | Cumulative collection | 21.50 |
| Example 8 | 60 − 80 − 100 | 2.5-1-0.5 | Cumulative collection | 19.98 |
| Comparative | 55 | 4 | Cumulative collection | 4.05 |
| Example 1 | | | | |
| Comparative | 75 | 4 | Cumulative collection | 19.83 |
| Example 2 | | | | |
| Comparative | 95 | 4 | Cumulative collection | 30.06 |
| Example 3 | | | | |

It can be seen from Table 1 that the extraction rate of the gradient hydrothermal method in the present disclosure was significantly improved compared with extraction at a low temperature (55° C.), and the extraction rate obtained by the cumulative collection mode was higher. In addition, although the extraction rate was slightly different from that obtained at 95° C., the gradient hydrothermal method avoids the damage of the collagen structure caused by direct long-time high-temperature cooking, which is more conducive to the use of collagen. In addition, the extraction rate of fish scale collagen according to the present disclosure fluctuates with the change of extraction time. Such a property provides an idea for reducing the extraction costs.

In the present disclosure, FTIR spectroscopy was used to measure the structural stability of the solid product.

Test method: The fish scale collagen sample and dried KBr powder were placed in a mortar according to a ratio of 1:100, thoroughly ground, tableted, and placed in a sample chamber. A spectrum measurement range of 4000-400 cm⁻¹ and a resolution of 8 cm⁻¹ were set.

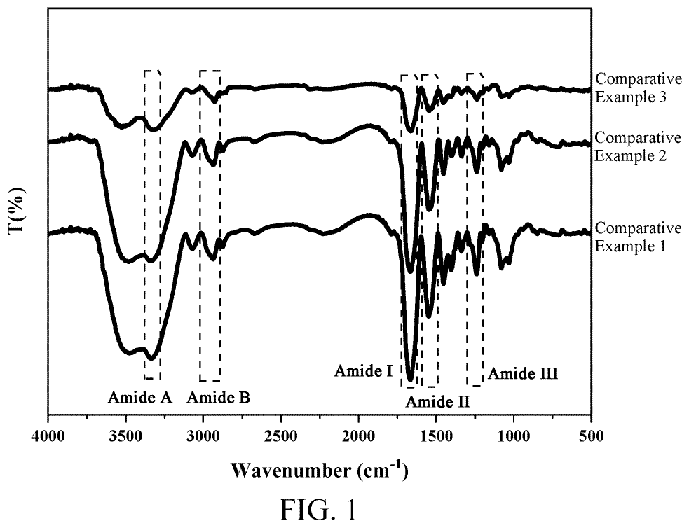

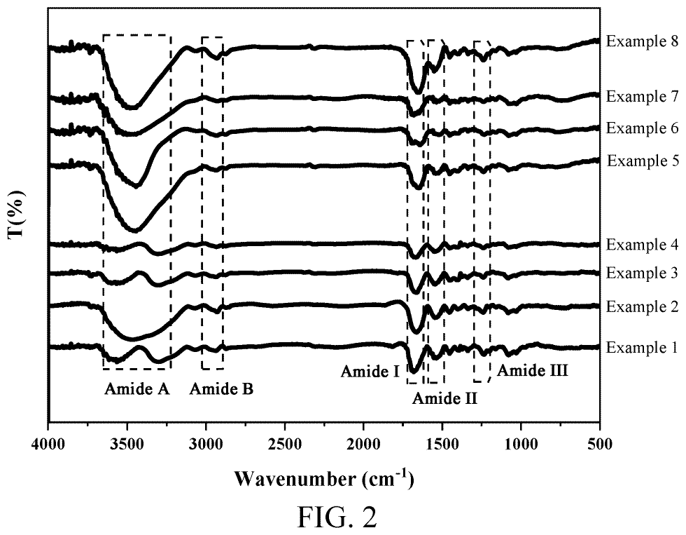

An infrared test was performed on the solid product prepared in the case of the present disclosure. The results are shown in FIG. 1 and FIG. 2. FIG. 1 is a diagram showing the FTIR spectroscopy results of fish scale collagen in comparative examples. FIG. 2 is a diagram showing the FTIR spectroscopy results of fish scale collagen in examples.

As can be seen from FIG. 1 and FIG. 2, the fish scale collagen extracted in the present disclosure has absorption peaks at around 3474 cm⁻¹, 2931 cm⁻¹, 1662 cm⁻¹, 1538 cm⁻¹, and 1244 cm⁻¹, which are characteristic absorption peaks of collagen. In addition, the infrared spectrum information of amide I band is often used to analyze the secondary structure of protein. As can be seen from FIG. 1, with the increase of extraction temperature, the peak intensity of the amide I band gradually weakened, indicating that the secondary structure of collagen was damaged. In FIG. 2, the collagen extracted by the gradient hydrothermal method had a high absorption intensity in the amide I band, indicating that the gradient hydrothermal method can retain a relatively complete protein structure.

In the present disclosure, the SDS-PAGE gel electrophoresis method was used to evaluate the size and distribution of protein molecular weight in the solid product.

Test method: 1) Preparation of resolving gel: A 7% resolving gel solution was prepared, poured into a mold to a certain height, overlaid with water, and allowed to stand at room temperature for polymerization. 2) Preparation of stacking gel: A 5% stacking gel solution was prepared. After the polymerization of the resolving gel solution, water was removed from the top of the resolving gel using a filter paper, then in the stacking gel solution was added, and a sample comb was inserted. 3) Preparation of sample or reference solution: 1 mg of the sample or standard was respectively taken, followed by addition of 100 μL of purified water. Equal volumes of the mixture and 2× sample buffer were mixed, heated in a 100° C. water bath for 5 to 10 minutes, and then centrifuged. 4) Electrophoresis: After the polymerization of the stacking gel, the sample comb was carefully pulled out, and the electrophoresis tank was filled with an electrode buffer. The sample and standard solution was added to the loading wells. The voltage of constant voltage electrophoresis was initially 80 V, and was adjusted to 100 V when entering the resolving gel. When the sample and standard solution sunk to the bottom of the gel, the electrophoresis ended. 5) Staining and destaining: The gel sheet was taken out and stained with an excess of Coomassie Blue for 1 to 2 hours. The staining solution was discarded, and the stained gel was destained multiple times with excess destaining solution until the gel background was clear. 6) Data analysis: Photographing and analysis were performed with a gel scanning system.

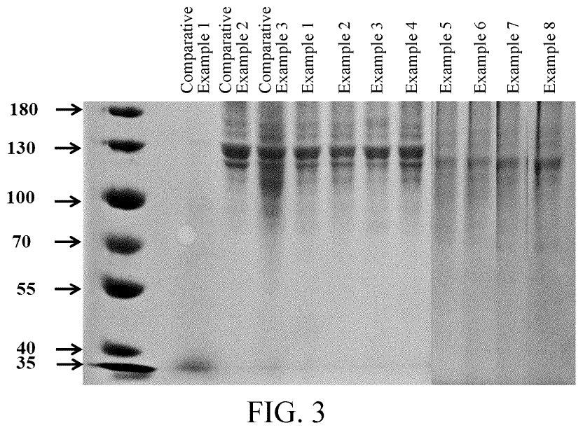

The solid products prepared in the examples and comparative examples of the present disclosure were tested by SDS-PAGE gel electrophoresis. The results are shown in FIG. 3. FIG. 3 is a graph showing the SDS-PAGE gel electrophoresis results of fish scale collagen. As can be seen from FIG. 3, under the same conditions, Comparative Example 1 (low-temperature extraction) failed to show significant protein bands, which was not conducive to collagen extraction. Collagen is thermal-sensitive to heat, and long-time high-temperature cooking will damage the structure of collagen, resulting in a dispersed molecular weight distribution. The fish scale collagen extracted by the gradient hydrothermal method had a uniform, stable, and concentrated molecular weight distribution in the respective temperature ranges, regardless of whether the temperature increase or decrease method was used and regardless of the collection mode used.

To sum up, the above information shows that the method provided by the present disclosure retains a relatively complete collagen structure while ensuring a high extraction rate, and is conducive to the use of collagen.

The above descriptions are merely several examples of the present disclosure, and are not to be construed as limiting the scope of the present disclosure. Any modifications and variations made without departing from the concept of the present disclosure fall within the scope of protection of the present disclosure.

What is claimed is:

1. A cost-effective method for extracting fish scale collagen having a large molecular weight, comprising:
1) performing pretreatment, comprising: collecting and packaging fish scales, followed by repeated frozen storage and thawing to obtain pretreated fish scales;
2) performing impurity protein removal, comprising: removing fish silver from the pretreated fish scales obtained in the step 1) and cleaning with pure water; and soaking the cleaned fish scales in a salt solution to remove impurity proteins, rinsing the fish scales with purified water until clear, followed by drying;
3) performing fish scale collagen extraction, comprising: mixing the dried fish scales obtained in the step 2) with purified water according to a ratio of 1:5 to 1:20, subjecting the mixture to hydrothermal extraction at 40° C. to 100° C. for 1 to 4 hours, performing solid-liquid separation, and collecting the solution as a fish scale collagen extract; and
4) pre-cooling and lyophilizing the fish scale collagen extract collected in the step 3) to obtain a solid fish scale collagen product.

2. The cost-effective method for extracting fish scale collagen having a large molecular weight according to claim 1, wherein: in the step 1), the frozen storage temperature of the packaged fish scales is −80° C. to −20° C.

3. The cost-effective method for extracting fish scale collagen having a large molecular weight according to claim 1, wherein: in the step 1), the temperature of thawing is 25° C. to 45° C., the time of thawing is 0.5 hours to 3 hours, and the number of times that the frozen storage and thawing operations are repeated is 1 to 3.

4. The cost-effective method for extracting fish scale collagen having a large molecular weight according to claim 1, wherein: in the step 2), the salt solution is any one of sodium chloride solution and sodium carbonate solution, the mass fraction of the salt solution is 5% to 20%, and the soaking time is 0.5 hours to 2 hours.

5. The cost-effective method for extracting fish scale collagen having a large molecular weight according to claim 4, wherein: in the step 2), the fish scales are dried at 20° C. to 65° C. for 3 hours to 24 hours.

6. The cost-effective method for extracting fish scale collagen having a large molecular weight according to claim 5, wherein: in the step 3), the hydrothermal extraction of fish scale collagen is a gradient hydrothermal extraction process, with gradient temperature intervals of 40° C. to 70° C., 70° C. to 90° C., and 90° C. to 100° C., and with the gradient temperature change being a gradient temperature increase or a gradient temperature decrease.

7. The cost-effective method for extracting fish scale collagen having a large molecular weight according to claim 1, wherein: in the step 3), the ratio of the fish scales to water is 1:5 to 1:10, the extraction time at 40° C. to 70° C. is 0.5 hours to 2 hours, the extraction time at 70° C. to 90° C. is 0.5 hours to 1 hour, and the extraction time at 90° C. to 100° C. is 0.5 hours to 2 hours.

8. The cost-effective method for extracting fish scale collagen having a large molecular weight according to claim 7, wherein: in the step 3), the solid-liquid separation is performed by filtration or centrifugation, and the fish scale collagen extract is collected by stages or cumulatively.

9. The cost-effective method for extracting fish scale collagen having a large molecular weight according to claim 8, wherein: in the step 4), the fish scale collagen extract is dried by any one of vacuum freeze-drying, spray freeze-drying, gel drying, and vacuum drying.

10. The cost-effective method for extracting fish scale collagen having a large molecular weight according to claim 1, wherein: the solid fish scale collagen product prepared by the method is in the form of powder, granules, or sponge.

11. The cost-effective method for extracting fish scale collagen having a large molecular weight according to claim 2, wherein: in the step 3), the ratio of the fish scales to water is 1:5 to 1:10, the extraction time at 40° C. to 70° C. is 0.5 hours to 2 hours, the extraction time at 70° C. to 90° C. is 0.5 hours to 1 hour, and the extraction time at 90° C. to 100° C. is 0.5 hours to 2 hours.

12. The cost-effective method for extracting fish scale collagen having a large molecular weight according to claim 3, wherein: in the step 3), the ratio of the fish scales to water is 1:5 to 1:10, the extraction time at 40° C. to 70° C. is 0.5 hours to 2 hours, the extraction time at 70° C. to 90° C. is 0.5 hours to 1 hour, and the extraction time at 90° C. to 100° C. is 0.5 hours to 2 hours.

13. The cost-effective method for extracting fish scale collagen having a large molecular weight according to claim 5, wherein: in the step 3), the ratio of the fish scales to water is 1:5 to 1:10, the extraction time at 40° C. to 70° C. is 0.5 hours to 2 hours, the extraction time at 70° C. to 90° C. is 0.5 hours to 1 hour, and the extraction time at 90° C. to 100° C. is 0.5 hours to 2 hours.

14. The cost-effective method for extracting fish scale collagen having a large molecular weight according to claim 6, wherein: in the step 3), the ratio of the fish scales to water is 1:5 to 1:10, the extraction time at 40° C. to 70° C. is 0.5 hours to 2 hours, the extraction time at 70° C. to 90° C. is 0.5 hours to 1 hour, and the extraction time at 90° C. to 100° C. is 0.5 hours to 2 hours.

15. The cost-effective method for extracting fish scale collagen having a large molecular weight according to claim 2, wherein: the solid fish scale collagen product prepared by the method is in the form of powder, granules, or sponge.

16. The cost-effective method for extracting fish scale collagen having a large molecular weight according to claim 3, wherein: the solid fish scale collagen product prepared by the method is in the form of powder, granules, or sponge.

17. The cost-effective method for extracting fish scale collagen having a large molecular weight according to claim 5, wherein: the solid fish scale collagen product prepared by the method is in the form of powder, granules, or sponge.

18. The cost-effective method for extracting fish scale collagen having a large molecular weight according to claim 6, wherein: the solid fish scale collagen product prepared by the method is in the form of powder, granules, or sponge.

19. The cost-effective method for extracting fish scale collagen having a large molecular weight according to claim 8, wherein: the solid fish scale collagen product prepared by the method is in the form of powder, granules, or sponge.

20. The cost-effective method for extracting fish scale collagen having a large molecular weight according to claim 9, wherein: the solid fish scale collagen product prepared by the method is in the form of powder, granules, or sponge.
